(19) United States
(12) Patent Application Publication
(10) Pub. No.: US 2026/0137437 A1
(43) Pub. Date: May 21, 2026

(54) POLYPECTOMY DEVICES

(71) Applicant: Boston Scientific Scimed, Inc., Maple Grove, MN (US)

(72) Inventors: Gonzalo Jose Saenz Villalobos, Alajuela (CR); Juan Pablo Ortiz Garcia, Heredia (CR); Katherin Narvaez Nuñez, Cartago (CR); Cristopher Oviedo Miranda, Alajuela (CR)

(73) Assignee: Boston Scientific Scimed, Inc., Maple Grove, MN (US)

(21) Appl. No.: 19/351,699

(22) Filed: Oct. 7, 2025

Related U.S. Application Data
(60) Provisional application No. 63/704,838, filed on Oct. 8, 2024.

(51) Int. Cl. A61B 18/08; A61B 18/00

(52) U.S. Cl. CPC A61B 18/082; A61B 2018/00077; A61B 2018/00083; A61B 2018/00136; A61B 2018/00482; A61B 2018/00601

(57) ABSTRACT

Polypectomy devices and methods for making and using polypectomy devices are disclosed. An example polypectomy device may include an elongate sheath extending from a proximal end region to a distal end, the elongate shaft including an inner reinforcing layer and an outer insulating layer, a shaft slidably disposed within the sheath, a handle coupled to the proximal end of the sheath, a longitudinal actuator disposed about the handle and secured to a proximal end of the shaft, and a snare coupled to a distal end of the shaft.

### CROSS-REFERENCE TO RELATED APPLICATIONS

This application claims the benefit of U.S. Provisional Patent Application Ser. No. 63/704,838 filed on Oct. 8, 2024, the disclosure of which is incorporated herein by reference.

### TECHNICAL FIELD

The present disclosure pertains to medical devices, and methods for manufacturing medical devices. More particularly, the present disclosure pertains to polypectomy devices including hot and cold capabilities.

### BACKGROUND

Medical devices, such as, but not limited to, endoscopes or other suitable introduction sheaths, may be employed for a variety of diagnostic and surgical procedures, such as, but not limited to, laparoscopy, arthroscopy, gynoscopy, thoracoscopy, cystoscopy, or the like. Many of these procedures are carried out for purposes of tissue resection, which generally includes removal of tissue of an organ or gland to treat tumors, infestations, or the like. Such procedures may be carried out by inserting an introduction sheath into a patient's body through a surgical incision or via a natural orifice and performing the procedure or operation.

Snares have been used in many medical procedures such as, but not limited to, endoscopic mucosal resection (EMR), endoscopic sub-mucosal resection (ESR), polypectomy, mucosectomy, or the like, for resecting tissue from a target site. A snare device may generally include a snare loop which engages the tissue intended to be resected. The snare loop may be controlled and operated at the proximal end of the device through a suitable actuation mechanism. Tissue resection may be performed using electrical current or electro-resectioning (e.g., hot) or without the application of an electrical current (e.g., cold). It may be desirable to provide a device which can be used effectively in hot resection or a cold resection.

### BRIEF SUMMARY

This disclosure provides design, material, manufacturing method, and use alternatives for medical devices.

In a first example, a polypectomy device may comprise an elongate sheath extending from a proximal end to a distal end, the elongate shaft including an inner reinforcing layer and an outer layer, a shaft slidably disposed within the sheath, a handle coupled to the proximal end of the sheath, a longitudinal actuator disposed about the handle and secured to a proximal end of the shaft, and a snare coupled to a distal end of the shaft.

Alternatively or additionally to any of the examples above, in another example, the inner reinforcing layer of the elongate sheath may comprise a helically wound coil.

Alternatively or additionally to any of the examples above, in another example, the helically wound coil may be closed pitch.

Alternatively or additionally to any of the examples above, in another example, the helically wound coil may be open pitch.

Alternatively or additionally to any of the examples above, in another example, the inner reinforcing layer of the elongate sheath may comprise a braided member.

Alternatively or additionally to any of the examples above, in another example, the inner reinforcing layer may comprise stainless steel.

Alternatively or additionally to any of the examples above, in another example, the outer layer may be an insulating layer.

Alternatively or additionally to any of the examples above, in another example, the outer layer may comprise a polymeric coating.

Alternatively or additionally to any of the examples above, in another example, the polypectomy device may further comprise an electrical connector electrically coupled with the shaft.

Alternatively or additionally to any of the examples above, in another example, the electrical connector may be configured to selectively supply an electrical current to the snare.

Alternatively or additionally to any of the examples above, in another example, the snare may be generally hexagonal.

Alternatively or additionally to any of the examples above, in another example, the snare may be generally oblong.

Alternatively or additionally to any of the examples above, in another example, the longitudinal actuator may be movable relative to the handle.

Alternatively or additionally to any of the examples above, in another example, a distal end of the inner reinforcing layer may extend distally beyond a distal end of the outer insulating layer.

Alternatively or additionally to any of the examples above, in another example, the polypectomy device may further comprise a tubular distal tip extending distally from a distal end of the inner reinforcing layer.

In another example, a polypectomy device may comprise an elongate sheath extending from a proximal end to a distal end. The elongate sheath may comprise an inner reinforcing layer, an outer insulating layer disposed over a radially outward surface of the inner reinforcing layer, and a tubular distal tip extending distally from a distal end of the inner reinforcing layer and free from the outer insulating layer. The polypectomy device may further comprise a shaft slidably disposed within the sheath, a handle coupled to the proximal end of the sheath, a longitudinal actuator disposed about the handle and secured to a proximal end of the shaft, the longitudinal actuator movable relative to the handle, an electrical connector configured to electrically couple the shaft with an external power source, and a snare coupled to a distal end of the shaft.

Alternatively or additionally to any of the examples above, in another example, the inner reinforcing layer may comprise a helically wound filar.

Alternatively or additionally to any of the examples above, in another example, the inner reinforcing layer may comprise a braided member.

Alternatively or additionally to any of the examples above, in another example, the inner reinforcing layer may be electrically conductive.

In another example, a polypectomy device may comprise an elongate sheath extending from a proximal end to a distal end. The elongate sheath may comprise an inner reinforcing layer comprising a stainless steel helically wound filar, an outer insulating layer disposed over a radially outward surface of the inner reinforcing layer, and a tubular distal tip extending distally from a distal end of the inner reinforcing layer and free from the outer insulating layer. The polypectomy device may further comprise a shaft slidably disposed within the sheath, a handle coupled to the proximal end of the sheath, a longitudinal actuator disposed about the handle and secured to a proximal end of the shaft, the longitudinal actuator movable relative to the handle, and a snare coupled to a distal end of the shaft.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an example medical device in a first configuration;

FIG. 2 is a bottom view of the illustrative medical device FIG. 1 in a second configuration;

FIG. 3 is a side view of a distal end region of a portion of the example medical device of FIG. 1;

FIG. 4 is a cross-sectional view of the distal end region of a portion of the example medical device of FIG. 3, taken at line 4-4 of FIG. 3;

FIG. 5 is a side view of a distal end region of a portion of an example medical device having an alternative configuration; and

FIG. 6 is a side view of a snare having an alternative configuration.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

This disclosure is now described with reference to an exemplary medical system including a snare. However, it should be noted that reference to this particular medical device is provided only for convenience and not intended to limit the disclosure. A person of ordinary skill in the art would recognize that the concepts underlying the disclosed devices and related methods of use may be utilized in any suitable device or procedure, medical or otherwise. This disclosure may be understood with reference to the following description and the appended drawings.

The term “distal” refers to a portion farthest away from a user when introducing a device into a patient. By contrast, the term “proximal” refers to a portion closest to the user when placing the device into the patient. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal”. Additionally, terms that indicate the geometric shape of a component/surface refer to exact and approximate shapes.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings. The drawings, which are not necessarily to scale, depict illustrative embodiments and are not intended to limit the scope of the invention.

Colonic polypectomy generally corresponds to the removal of colorectal polyps, for example, in order to prevent them from turning cancerous. Some polypectomy systems may include a snare that is engaged with a polyp. The polyp may be severed with the snare. During a polypectomy procedure it may be considered normal for the physician to encounter different polyp sizes. For example, polyps may range from about 1 millimeter (mm) to the centimeter range. In some cases, the size of the polyp may factor into the clinician's decision when determining which tool to use to remove the polyp. To perform a polypectomy using snare techniques, a clinician may take into consideration snare features such as, but not limited to, loop size, loop shape configuration, stiffness, and/or hot/cold polypectomy. Thus, it may be desirable to provide a polypectomy device which can perform hot or cold polypectomy, has different loop sizes, and/or a higher wire flexibility. While the disclosure is described with respect to a snare, the principles of this disclosure may be applied to other end effectors or other devices, such as, but not limited to, graspers, biopsy jaws, scissors, or the like.

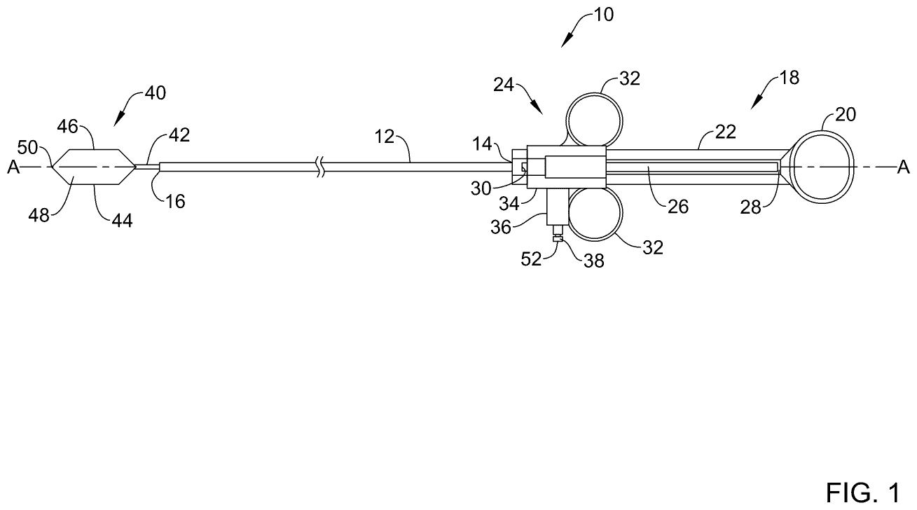

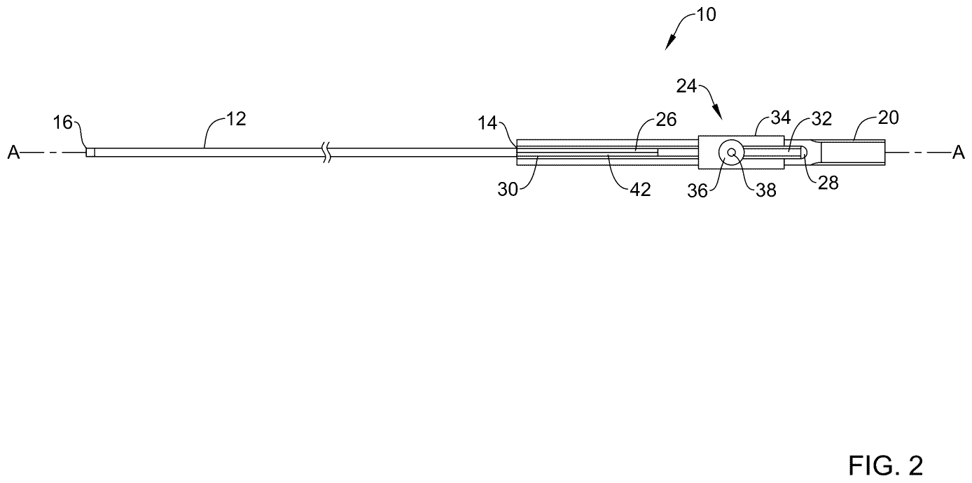

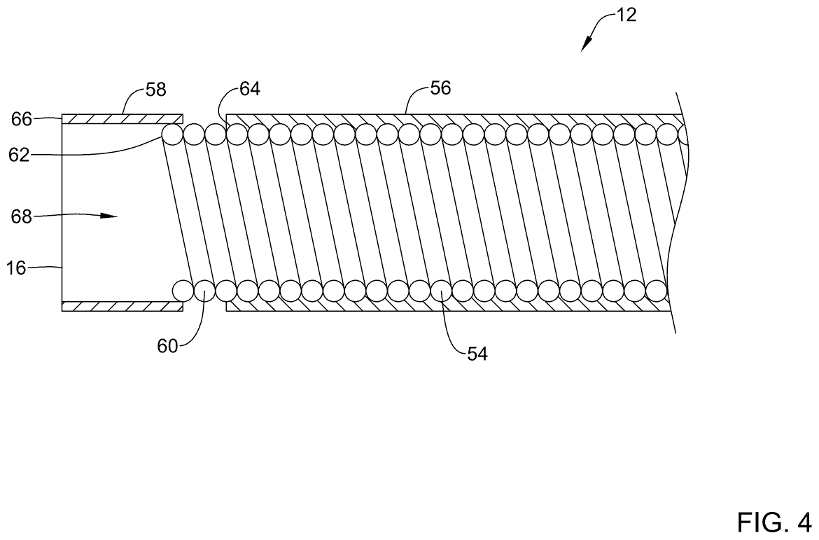

Referring to FIGS. 1 and 2 which illustrate a side view of an illustrative polypectomy device 10 in a first configuration and a bottom view of the illustrative polypectomy device 10 in a second configuration, respectively, the polypectomy device 10 may include a longitudinal axis A extending from a proximal end to a distal end of the device 10. The polypectomy device 10 may include a sheath or elongate shaft 12 extending from a proximal end 14 to a distal end 16. The sheath 12 may include a lumen 68 (see, for example, FIG. 4) extending from the proximal end 14 to the distal end 16 thereof. The proximal end 14 of the sheath 12 may be fixedly secured to a handle 18. In some embodiments, the proximal end 14 of the sheath 12 may be secured to a distal end of the handle 18. In other embodiments, the proximal end 14 of the sheath 12 may extend proximal to a distal end of the handle 18 (e.g., may extend into a lumen of the handle 18). The sheath 12 and the handle 18 may have a variety of different forms and/or configurations. In one example, the sheath 12 may have a length that is suitable to extend through an endoscope to position the sheath 12 within a body lumen. This may include a body lumen along the digestive tract such as along the small intestine and/or colon. Other body lumens may also be accessed with the sheath 12.

The handle 18 may be positioned towards the proximal end of the polypectomy device 10 and may be configured to remain outside the body. The handle 18 may include a finger opening 20 and an elongate shaft 22. During use, the finger opening 20 may receive a user's thumb (or another finger). The elongate shaft 22 may have a circular, oval, or other exterior shape and may define an interior lumen (not explicitly shown). In some embodiments, the lumen may extend through an entirety of a length of the elongate shaft 22. However, this is not required. In some embodiments, the lumen may terminate distal to a proximal end of the elongate shaft 22. The handle 12 may further include a longitudinal actuator 24. The longitudinal actuator 24 may be configured to be axially displaced relative to the handle 18 along the longitudinal axis A. For example, the longitudinal actuator 24 may be slidably disposed within a slot 26 of the elongate shaft 22. The slot 26 may extend from a proximal end 28 to a distal end 30 generally parallel to the longitudinal axis A of the polypectomy device 10. The slot 26 may extend less than an entirety of the length of the elongate shaft 22 of the handle 18. It is contemplated that the proximal end 28 and/or distal end 30 of the slot 26 may provide mechanical stops which limit proximal and/or distal movement of the longitudinal actuator 24 and prevent the longitudinal actuator 24 from being accidentally disengaged from the handle 18. While not explicitly shown, a portion of the longitudinal actuator 24 may extend radially inwards into the slot 26 such the longitudinal actuator 24 engages and/or slides along the slot 26.

The longitudinal actuator 24 may include one or more finger openings 32, an elongate shaft 34, and a lateral extension 36 housing an electrical connector 38. During use, the one or more finger openings 32 may each receive a user's finger. However, this is not required. The elongate shaft 34 may be a hollow shaft that extends along the longitudinal axis A and is disposed around an outer surface of the elongate shaft 22 of the handle 18. The longitudinal actuator 24 may include a radially inward extending portion (not explicitly shown) configured to extend into and/or engage with the slot 26 of the handle 18. The longitudinal actuator 24 may be axially displaced (e.g., proximally retracted and/or distally advanced) relative to the handle 18 in a direction generally parallel to the longitudinal axis A. However, engagement of the radially inwards extending portion with the slot 26 may prevent the longitudinal actuator 24 from rotating relative to the handle 18. In FIG. 1, the longitudinal actuator 24 is in a distally advanced configuration while in FIG. 2 the longitudinal actuator 24 is in a proximally retracted configuration. The longitudinal actuator 24 may be positioned anywhere in between a proximal-most location and a distal-most location along the slot 26, as desired.

The polypectomy device 10 may further include a snare 40. The snare 40 may be connected to or continuous with a shaft or wire 42. Generally, the snare 40 may be at or adjacent to a distal end of the shaft 42. The snare 40 and shaft 42 may be slidably disposed within the lumen 68 of the sheath 12. For example, the snare 40 and shaft 42 may be distally advanced via the longitudinal actuator 24 to position the snare 40 distal to the distal end 16 of the sheath 12 (FIG. 1) or proximally retracted via the longitudinal actuator 24 to position the snare 40, at least partially, within the lumen 68 of sheath 12 (FIG. 2).

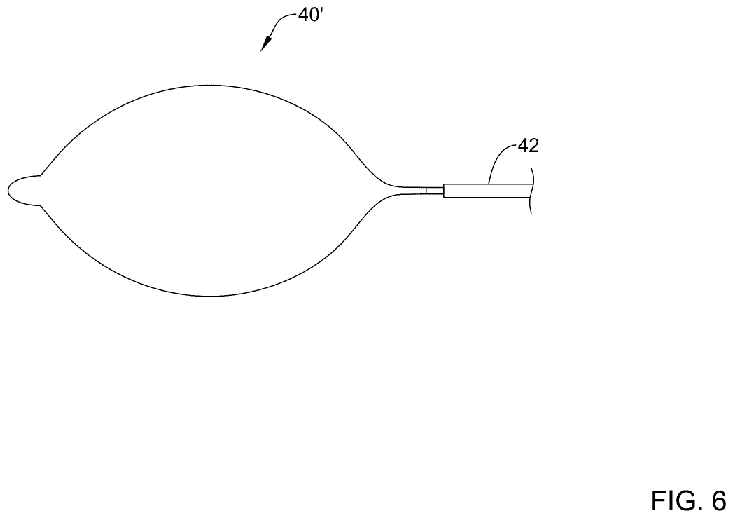

The snare 40 may include a first side 44 and a second side 46 which together generally form a loop defining an enclosed region 48. The first and second sides 44, 46 may connect and/or contact each other at a proximal end of the snare 40 and at a distal tip 50. In one example, the snare 40 may have a generally hexagonal shape with a curved distal tip 50. However, this is not required. FIG. 6 is a side view of a snare 40′ having a generally oblong or a vesical piscis shape (e.g., a football shape). Other shapes are also contemplated. Returning to FIGS. 1 and 2, the snare 40 may take other shapes, regular or irregular, as desired. In some cases, regular shapes may include irregularities near the proximal or distal ends or anywhere therebetween. The enclosed region 48 defined by the snare 40 may be an opening that receives tissue during a medical procedure.

The first and second sides 44, 46 of the snare 40 may be formed from two separate wires connected to or contacting each other at their proximal and distal ends or from a single wire. If the snare 40 is formed from a single wire, the single wire may include a connection or contact between ends of the wire proximate the proximal end of snare 40 and the distal tip of snare 40 may be continuous and formed from the unbroken wire. As used herein, the term “wire” means any elongated member capable of translating within the outer sheath 12. A single wire may be formed from a single filament and/or a single monolith of material (e.g., a singular polymer, a combination or blend of polymers formed into a single filament/monolith of material, a singular metal or metal alloy (e.g., a nickel-titanium alloy), a combination of metals and/or alloys formed into a single filament/monolith of material, etc.). In other instances, “wire” may refer to a bundle of individual wires or elongated members that are electrically or physically connected and therefore act as a single wire, such that a snare 40 formed of a single “wire” may be formed from a bundle of continuous wires, a cable, or any other elongate member. A snare 40 formed of two “wires” therefore may be formed by two separate bundles of wires that are connected to and/or contact each other proximate the proximal end of the snare 40 and at the distal tip 50.

The snare 40 may have an enclosed region 48 or opening size (e.g., inner diameter or width) of about 10 to 55 millimeters (mm) (about 0.39 inches to about 2.17 in). The wire forming the snare 40 may have a diameter of about 0.13 mm to about 1.27 mm (0.005-0.050 inches), or about 0.20 mm to about 1.02 mm (0.008-0.040 inches). The diameter of wire forming the snare 40 may be chosen based on the length of snare. For example, a snare 40 that is about 30 mm in length may utilize a wire having a diameter of about 0.64 mm (0.025 inches). This is just one example.

The two sides 44, 46 of the snare 40 may join together and extend proximally to form the shaft 42. Said differently, the snare 40 and the shaft 42 may be formed from a single monolithic structure. In an alternative example, the two sides 44, 46 of the snare 40 may end at a location proximal to the snare 40 but may be electrically connected to and secured to a separate shaft 42. The shaft 42 may extend proximally through the sheath 12 to the longitudinal actuator 24. The shaft 42 may be removably or fixedly secured to the longitudinal actuator 24 such that proximal and/or distal movement of the longitudinal actuator 24 is translated to the shaft 42 and the snare 40. For example, the longitudinal actuator 24 may be fixed relative to the shaft 42 by crimping, adhesive, or any other suitable attachment method. In some cases, the shaft 42 may be coupled to the longitudinal actuator 24 such that electrical energy may be transmitted from an energy source (not explicitly shown) through the electrical connector 38 and to the shaft 42 and snare 40.

The electrical connector 38 may extend from a first end 52 exterior to the lateral extension 36 to a second end proximate to an interior of the handle 18 and/or interior of the longitudinal actuator 24. In some cases, the second end of the electrical connector 38 may be electrically coupled to the longitudinal actuator 24 which in turn is electrically coupled to the shaft 42. In other examples, the second end of the electrical connector 38 may be electrically coupled directly to the shaft 42. The first end 52 of the electrical connector 38 may be connected through wires or other mechanisms (not explicitly shown) to a source of electricity, such as, but not limited to, a generator (not explicitly shown).

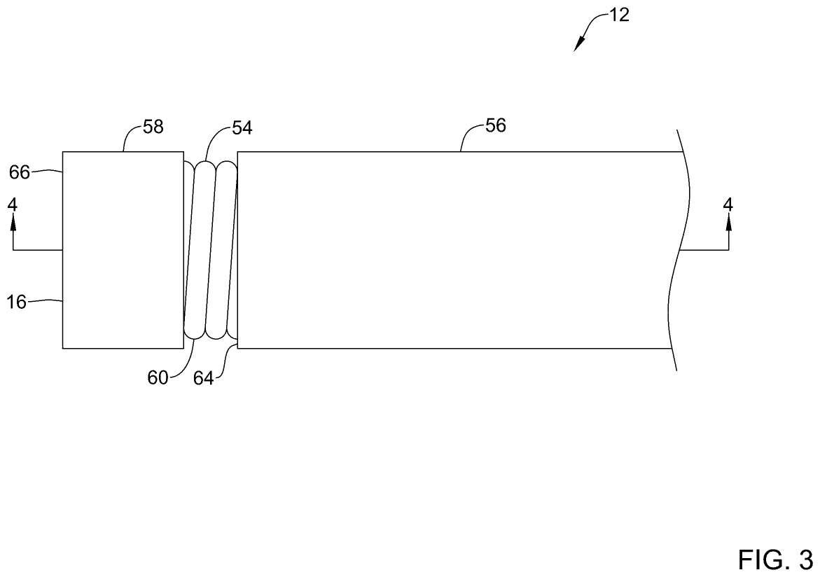

In some cases, it may be difficult to provide a polypectomy device 10 which can efficiently perform either hot or cold resection efficiently. Hot snaring may use electrical energy to heat the snare 40 for electrocautery. Cold snaring may use only mechanical actuation cutting. These techniques may require conflicting features. For example, hot resection may require the sheath 12 to be electrically insulated while cold snaring may require the sheath 12 to have a high compressive strength to prevent buckling or bending of the sheath 12. FIG. 3 is a side view of a distal end region of an illustrative sheath 12 which may provide high compressive strength for cold snaring as well as electrical insulation for hot snaring. FIG. 4 is a cross-sectional view of the distal end region of the sheath 12, taken at line 4-4 of FIG. 3. Generally, the sheath 12 may include an inner layer 54 and an outer layer 56. The inner layer 54 may provide compressive strength to the sheath 12 while the outer layer 56 may provide electrical insulation.

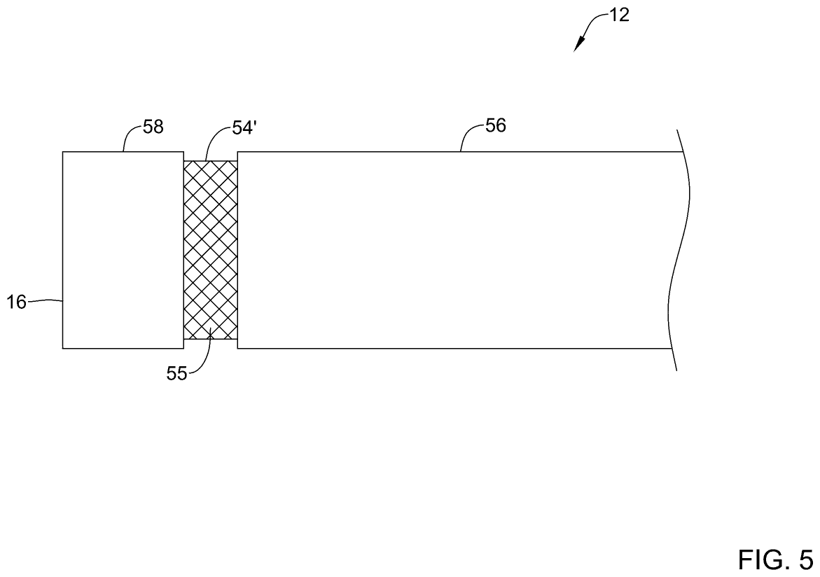

In some embodiments, the inner layer 54 may be a support layer or reinforcing layer configured to provide compressive strength to the sheath 12. In the illustrated embodiment, the inner layer 54 may take the form of a coil defining a lumen 68. However, in other embodiments, the inner layer 54 may take the form of a braid or other support member. For example, FIG. 5 illustrates a side view of an illustrative sheath 12 in which the inner layer 54′ is a braid. The braid may be formed from any number or configuration of strands or ribbons 55 and braided or woven at any desired density.

Returning to FIGS. 3 and 4, the lumen 68 may extend from the proximal end 14 to the distal end 16 of the sheath 12. While not explicitly shown in FIGS. 3 and 4, the snare 40 and the shaft 42 may be movably received or disposed within the lumen 68. The inner layer 54 may include one or more filars 60. For purposes of this discussion, a “filar” may be understood as a wire or wires that are helically wound into a coiled configuration in order to form or otherwise define a coil. The filar 60 is not limited to a circular cross-sectional shape. The filar 60 may take other cross-sectional shapes as desired, such as, but not limited to, square, rectangular, polygonal, triangular, oblong, irregular, or the like.

As can be seen in FIG. 4, the filar 60 may have a uniform cross-sectional diameter and pitch. While a uniform cross-sectional diameter is shown in FIG. 4, it is contemplated that the cross-sectional diameter of the filar 60 may vary along the length of filar 60 and/or along a length of the inner layer 54. Additionally, the inner layer 54 may be configured to have an open pitch, a closed pitch or combinations thereof. For example, FIGS. 3 and 4 show the filar 60 arranged such that there is no space between the adjacent individual windings. The absence of space between the windings may be referred to as a “closed” pitch configuration. A closed pitch configuration may be desirable to provide increased column strength to the sheath 12. An open pitch configuration may be defined as space existing between adjacent windings of the filar 60. Further, characteristics such as the filar cross-sectional dimension and/or shape, material, orientation, and spacing may contribute to the overall configuration and performance (e.g., flexibility, pushability, trackability, column stiffness, etc.) of the inner layer 54.

Proximal retraction of the snare 40 into the sheath 12 may exert a compressive force on the sheath 12 as the snare 40 is pulled into and compressed within the lumen 68 of the sheath 12. It is contemplated that the inner layer 54 may be formed from a material which resists deformation due to thermal effects and/or compression due to actuation of the snare 40. For example, the inner layer 54 of the sheath 12 may be formed from a material which resists bending or deformation as the snare 40 is proximally retracted into the sheath 12. In some embodiments, the inner layer 54 may be formed from a metal or metal alloy such as, but not limited to, stainless steel, nickel-titanium alloys, nickel-chromium-molybdenum alloys, other nickel alloys, titanium, or the like. It is contemplated that the material properties which improve the compressive strength of the sheath 12 may also have a higher axial and lateral load-carrying capacity to improve the efficiency of the polypectomy device 10 when used without electrical current. Further, the inner layer 54 may be electrically conductive such that the inner layer 54 and/or the distal tip 58 may be used as a marking tool.

It is contemplated that forming the inner layer 54 from a stainless steel coil or braid may improve the flexibility of the sheath 12. For example, a coil or braid formed from stainless steel (or other material which resists compression) may increase the flexibility of the sheath 12 relative to a sheath formed from plastic or polymers. The coil or braid may allow the sheath 12 to be temporarily deformed while allowing the sheath 12 to resist mechanical deformation.

The inner layer 54 may extend from a proximal end (not explicitly shown) proximate to the proximal end 14 of the sheath 12 to a distal end 62. In some embodiments, the distal end 62 of the inner layer 54 may be proximal to the distal end 16 of the sheath 12. However, this is not required. In some examples, the distal end 62 of the inner layer 54 may align with or be generally adjacent to the distal end 16 of the sheath 12. In some embodiments, the proximal end of the inner layer 54 may be distal to the proximal end 14 of the sheath 12.

The outer layer 56 may be a coating or jacket disposed over a radially outward outer surface of the inner layer 54. In some embodiments, the outer layer 56 may be formed from an electrically insulating material, such as a polymeric material. Some illustrative electrically insulating materials may include, but are not limited to, polyethylene terephthalate (PET), polyamides, polyesters, polyurethanes, fluoropolymers, or polyimides. These are just examples, other insulating materials, polymeric or otherwise, are also contemplated. A thickness of the outer layer 56 may vary depending on the lubricity of the material and/or the insulative properties of the material (e.g., how well the material electrically isolates). The outer layer 56 may extend from a proximal end (not explicitly shown) proximate to the proximal end 14 of the sheath 12 to a distal end 64. In some embodiments, the distal end 64 of the outer layer 56 may be proximal to the distal end 16 of the sheath 12. However, this is not required. In some examples, the distal end 64 of the outer layer 56 may align with or be generally adjacent to the distal end 16 of the sheath 12. In yet other examples, the distal end 64 of the outer layer 56 may align with or be generally adjacent to the distal end 62 of the inner layer 54. In some embodiments, the proximal end of the inner layer 56 may be distal to the proximal end 14 of the sheath 12. It is contemplated that the outer layer 56 may electrically insulate the sheath 12 such that electrical energy is not inadvertently transferred to regions of the body outside of the target tissue.

In some embodiments, an optional tubular distal tip 58 or cannula may be secured to the inner layer 54 proximate the distal end 62 thereof. The distal tip 58 may extend distally beyond the distal end 62 of the inner layer 64. The distal tip 58 may be a tubular member configured to perform a marking step prior to performing the cutting step. In some examples, the distal end 66 of the distal tip 58 may be brought into contact with the tissue adjacent to the polyp (or other tissue to be resected) and suction applied. The suction may leave a visual mark on the tissue to help identify the tissue to be resected. Other marking techniques may be used, as desired. It is contemplated that the distal tip 58 may be formed from a metal or metal alloy and welded to the distal end region of the inner layer 54. In some cases, the outer diameter of the distal tip 58 may be greater than an outer diameter of the inner layer 54 and/or outer layer 56. In yet other embodiments, the distal tip 58 may be omitted and a length of the inner layer 54 extending proximally from the distal end 62 thereof may be free from the outer layer 56 such that the inner layer 54 may be used to perform the marking step.

As described herein, the inner layer 54 may resist bending and/or deformation as the snare 40 is distally advanced from the sheath 12 or proximally retracted into the sheath 12. Further, forming the inner layer 54 from a metal or metal alloy may allow heat to be generated at the snare 40 without risk of melting or deforming a polymeric shaft. The outer layer 56 may provide electrical insulation to prevent accidental burns or tissue damage to surrounding tissues.

During a medical procedure, the polypectomy device 10 may be used to resect tissue. The polypectomy device 10 may be inserted into a patient with the snare 40 in a retracted position within the lumen 68 of the sheath 12, as shown in FIG. 2. In one example, the sheath 12 containing the snare 40 may be inserted through a working channel of an endoscope or other tool. However, this is not required. When the snare 40 is in a proximally retracted configuration, the longitudinal actuator 24 may be in a proximal position relative to the handle 18. For example, the longitudinal actuator 24 is positioned closer to the proximal end of the handle 18 when the snare 40 is proximally retracted into the lumen 68 of the sheath 12. Further, when the snare 40 is disposed within the lumen 68 of the sheath 12, the snare 40 may be in a radially compressed configuration. When the user desires to extend the snare 40, the user may distally advance the longitudinal actuator 24. As the longitudinal actuator 24 is distally advanced, the shaft 42 and the snare 40 are also distally advanced causing the snare 40 to extend distally beyond the distal end 16 of the sheath 12. The longitudinal actuator 24 may be distally advanced until a portion of the longitudinal actuator 24 abuts or contacts a distal end 30 of the slot 26.

The slot 26 may have a length such that an entirety of the loop of the snare 40 may be advanced distally beyond a distal end 16 of the sheath 12. This may allow the snare 40 to move from a radially collapsed delivery configuration to an expanded use configuration. However, in some cases, the snare 40 may not be distally advanced to the distalmost configuration. The user may position the snare 40 around the tissue to be resected. If the user desires to conduct hot resection or electro-resection, the electricity source can be turned on to send electrical energy through the connector 38 to the shaft 42 and snare 40 which are all electrically connected. The user may proximally retract the longitudinal actuator 24 during application of electrical energy to the tissue such that the snare 40 tightens around and cuts the tissue to be removed. Electrical energy may be continued to be applied to the tissue even when the snare 40 is moved proximally or distally. Once cut, the tissue may be removed from the patient using tools through a working channel of an endoscope or other medical device.

If a user desires to conduct cold resection (e.g., without the application of electrical energy), the steps may be similar to hot resection with the omission of activating the energy source. For example, the snare 40 may be distally advanced to and positioned around the tissue to be resected. The user may proximally retract the longitudinal actuator 24 (with no electrical energy) such that the snare 40 tightens around and cuts the tissue to be removed. Once cut, the tissue may be removed from the patient using tools through a working channel of an endoscope or other medical device.

The materials that can be used for the various components of polypectomy device 10 (and/or other polypectomy devices disclosed herein) and the various wires, snares, sheaths, etc. disclosed herein may include those commonly associated with medical devices. For simplicity purposes, the following discussion makes reference to sheath 12 and other components of device 10. However, this is not intended to limit the devices and methods described herein, as the discussion may be applied to other similar tubular members and/or components of the devices disclosed herein.

Sheath 12 and/or other components of device 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of device 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of device 10 in determining its location. Some examples of radiopaque materials can include, but are not limited to, gold, platinum, palladium, tantalum, tungsten alloy, polymer material loaded with a radiopaque filler, polymer material having covalently attached iodine groups, and the like. Additionally, other radiopaque marker bands and/or coils may also be incorporated into the design of device 10 to achieve the same result.

In some embodiments, a degree of Magnetic Resonance Imaging (MRI) compatibility is imparted into device 10. For example, device 10, or portions thereof, may be made of a material that does not substantially distort the image and create substantial artifacts (e.g., gaps in the image). Certain ferromagnetic materials, for example, may not be suitable because they may create artifacts in an MRI image. Device 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A polypectomy device, comprising:
an elongate sheath extending from a proximal end to a distal end, the elongate shaft including an inner reinforcing layer and an outer layer;
a shaft slidably disposed within the sheath;
a handle coupled to the proximal end of the sheath;
a longitudinal actuator disposed about the handle and secured to a proximal end of the shaft; and
a snare coupled to a distal end of the shaft.

2. The polypectomy device of claim 1, wherein the inner reinforcing layer of the elongate sheath comprises a helically wound coil.

3. The polypectomy device of claim 2, wherein the helically wound coil is closed pitch.

4. The polypectomy device of claim 2, wherein the helically wound coil is open pitch.

5. The polypectomy device of claim 1, wherein the inner reinforcing layer of the elongate sheath comprises a braided member.

6. The polypectomy device of claim 1, wherein the inner reinforcing layer comprises stainless steel.

7. The polypectomy device of claim 1, wherein the outer layer is an insulating layer.

8. The polypectomy device of claim 1, wherein the outer layer comprises a polymeric coating.

9. The polypectomy device of claim 1, further comprising an electrical connector electrically coupled with the shaft.

10. The polypectomy device of claim 9, wherein the electrical connector is configured to selectively supply an electrical current to the snare.

11. The polypectomy device of claim 1, wherein the snare is generally hexagonal.

12. The polypectomy device of claim 1, wherein the snare is generally oblong.

13. The polypectomy device of claim 1, wherein the longitudinal actuator is movable relative to the handle.

14. The polypectomy device of claim 1, wherein a distal end of the inner reinforcing layer extends distally beyond a distal end of the outer insulating layer.

15. The polypectomy device of claim 1, further comprising a tubular distal tip extending distally from a distal end of the inner reinforcing layer.

16. A polypectomy device, comprising:
an elongate sheath extending from a proximal end to a distal end, the elongate sheath comprising:
an inner reinforcing layer;
an outer insulating layer disposed over a radially outward surface of the inner reinforcing layer; and
a tubular distal tip extending distally from a distal end of the inner reinforcing layer and free from the outer insulating layer;
a shaft slidably disposed within the sheath;
a handle coupled to the proximal end of the sheath;
a longitudinal actuator disposed about the handle and secured to a proximal end of the shaft, the longitudinal actuator movable relative to the handle;
an electrical connector configured to electrically couple the shaft with an external power source; and
a snare coupled to a distal end of the shaft.

17. The polypectomy device of claim 16, wherein the inner reinforcing layer comprises a helically wound filar.

18. The polypectomy device of claim 16, wherein the inner reinforcing layer comprises a braided member.

19. The polypectomy device of claim 16, wherein the inner reinforcing layer is electrically conductive.

20. A polypectomy device, comprising:
an elongate sheath extending from a proximal end to a distal end, the elongate sheath comprising:
an inner reinforcing layer comprising a stainless steel helically wound filar;
an outer insulating layer disposed over a radially outward surface of the inner reinforcing layer; and
a tubular distal tip extending distally from a distal end of the inner reinforcing layer and free from the outer insulating layer;
a shaft slidably disposed within the sheath;
a handle coupled to the proximal end of the sheath;
a longitudinal actuator disposed about the handle and secured to a proximal end of the shaft, the longitudinal actuator movable relative to the handle; and
a snare coupled to a distal end of the shaft.
